(19) United States
(12) Patent Application Publication
(10) Pub. No.: US 2018/0029955 A1
(43) Pub. Date: Feb. 1, 2018

(54) Method and apparatus for producing hydrocarbons

(71) Applicant: Linde Aktiengesellschaft, München (DE)

(72) Inventors: Heinz Zimmermann, München (DE); Florian Penner, Geretsried (DE)

(21) Appl. No.: 15/551,252

(22) PCT Filed: Feb. 16, 2016
(86) PCT No.: PCT/EP2016/053282

§ 371 Date: Aug. 15, 2017

(30) Foreign Application Priority Data
Feb. 16, 2015 (EP) 15155233.8

(51) Int. Cl. C07C 2/84; C07C 5/327; C07C 4/04

(52) U.S. Cl. CPC C07C 2/84; C07C 4/04; C07C 5/327

(57) ABSTRACT

A method (100) for producing hydrocarbons is proposed, in which one or more steam cracking feed streams (a) which predominantly or exclusively contain hydrocarbons with two or more carbon atoms are subjected to one or more steam cracking steps (10), thus obtaining one or more steam cracking discharge streams (b), and wherein one or more reaction feed streams (t, u) which predominantly or exclusively contain methane are subjected to one or more steps (60) for the oxidative coupling of methane, thus obtaining one or more reaction discharge streams (v) which contain ethane, while a separation discharge stream (m) which predominantly or exclusively contains ethane is formed using fluid from the steam cracking discharge stream or streams (b). In the proposed method, it is provided that fluid from the reaction discharge stream or streams (v) is subjected to one or more thermal cracking steps (70) which are subsequent to the step or steps (60) for the oxidative coupling of methane, and in which the ethane which is present in the fluid from the reaction discharge stream or streams (v) is at least partially reacted to form ethylene, under the influence of waste heat from the step or steps (60) for the oxidative coupling of methane, and that fluid (w) from the separation discharge stream (m) is fed into the subsequent thermal cracking step or steps (70), wherein the step or steps (60) for the oxidative coupling of methane and the subsequent thermal cracking step or steps (70) are carried out in a joint reactor and wherein the transfer of heat into the thermal cracking step or steps (70) that follow takes place by convection.

The invention relates to a method for producing hydrocarbons according to the pre-characterising clause of claim 1.

### PRIOR ART

Methods and apparatus for steam cracking hydrocarbons are known and are described for example in the article “Ethylene” in Ullmann's Encyclopedia of Industrial Chemistry, online since 15 Apr. 2007, DOI 10.1002/14356007.a10_045.pub2. In steam cracking, hydrocarbons are cracked in the presence of steam, typically at temperatures of 800 to 860° C. over retention times of about 0.1 to 0.8 seconds.

Steam cracking processes are carried out on a commercial scale almost exclusively in heated tubular reactors. As is generally known, steam cracking processes produce gas mixtures which contain by-products in addition to the actual target compounds. In particular, typically fairly large amounts of methane and hydrogen are formed in steam cracking processes. These components of the so-called heating gas fraction are conventionally used individually or together as heating gas for heating the above-mentioned tubular reactors.

However, at the same time, the material use of methane by value-adding reactions is of great interest from an economic point of view. Thus, methods for producing higher hydrocarbons from methane by oxidative methane coupling (Oxidative Coupling of Methane, OCM) are currently in intensive development. Oxidative coupling of methane is the direct reaction of methane in an oxidative, heterogeneously catalysed process to form higher hydrocarbons. Such methods appear to be highly promising, particularly for the manufacture of ethylene.

For further details of the oxidative coupling of methane, reference may be made to the relevant specialist literature, for example Zavyalova, U. et al.: Statistical Analysis of Past Catalytic Data on Oxidative Methane Coupling for New Insights into the Composition of High-Performance Catalysts, Chem Cat Chem 3, 2011, 1935-1947. From U.S. Pat. No. 5,254,781 A, e.g., a method is known that comprises oxidative methane coupling and steam cracking. From U.S. Pat. No. 5,118,898 A and EP 2 711 348 A further methods for oxidative methane coupling are known.

Combined processes which include steam cracking processes and processes for preparing higher hydrocarbons from methane by oxidative methane coupling are also known in principle. There is a need for an improved integration of materials in such processes. In particular, compounds produced in one of the two processes should be utilised in the other process wherever possible, if this is beneficial from the point of view of economy and/or energy efficiency.

### DISCLOSURE OF THE INVENTION

This problem is solved by a method for producing hydrocarbons having the features of claim 1. Embodiments are the subject of the dependent claims and the description that follows.

Before the features and advantages of the present invention are discussed, their basis and the terms used will be explained.

As already mentioned, steam cracking processes are carried out on a commercial scale almost exclusively in tubular reactors in which individual reaction tubes (in the form of coiled tubes, so-called coils) or groups of corresponding reaction tubes can be operated even under different cracking conditions. Reaction tubes or sets of reaction tubes operated under identical or comparable cracking conditions and possibly also tubular reactors operated under uniform cracking conditions are also referred to as “cracking furnaces”. A cracking furnace in the terminology used here is thus a construction unit used for steam cracking which subjects a furnace feed to identical or comparable cracking conditions. A steam cracking unit (also referred to as an “olefin plant”) may comprise one or more cracking furnaces of this kind. A “steam cracking step” is carried out in one or more cracking furnaces, in the terminology of the present application.

The term “furnace feed” here denotes one or more liquid and/or gaseous streams which are supplied to one or more cracking furnaces. Streams obtained from a corresponding steam cracking process, as explained hereinafter, may also be recycled into one or more cracking furnaces and used again as a furnace feed. Suitable furnace feeds include a number of hydrocarbons and hydrocarbon mixtures from ethane to gas oil up to a boiling point of typically 600° C.

A furnace feed may consist of a so-called “fresh feed”, i.e. a feed which is prepared outside the apparatus and is obtained for example from one or more petroleum fractions, petroleum gas and/or petroleum gas condensates. A furnace feed may also consist of one or more so-called “recycle streams”, i.e. streams that are produced in the apparatus itself and recycled into a corresponding cracking furnace. A furnace feed may also consist of a mixture of one or more fresh feeds with one or more recycle streams.

The furnace feed is at least partly reacted in the respective cracking furnace and leaves it as a so-called “crude gas” which can be subjected to after-treatment steps. These encompass, first of all, processing of the crude gas, for example by quenching, cooling and drying, so as to obtain a so-called “cracked gas”. Occasionally the crude gas is also referred to as cracked gas.

The term “steam cracking feed stream” is also used hereinafter as a general designation for a mixture of components supplied to one or more cracking furnaces, i.e. one or more furnace feeds as explained above, while the term “steam cracking discharge stream” is also used hereinafter as a general designation for a gas mixture taken from one or more cracking furnaces.

As already mentioned, a steam cracking discharge stream is a mixture of hydrocarbons which contains by-products in addition to the desired target compounds. A steam cracking discharge stream is therefore typically at least partly separated into fractions. This may be done using differently configured separation sequences, to which a steam cracking discharge stream is subjected, for example, after a so-called crude gas compression and other processing steps. Corresponding separation sequences are also explained in the above-mentioned article “Ethylene” in Ullmann's Encyclopedia of Industrial Chemistry.

By a “separation discharge stream” is meant any stream produced in a corresponding separation sequence. Where it states hereinafter that “fluid” from a stream (particularly fluid from a separation discharge stream) is used for specific purposes, this may include the use of the entire stream or, alternatively, only part of it or only specific components. If a separation discharge stream or another (substance) stream “predominantly” contains one or more components, within the terminology of the present application, the component(s) is or are present in a proportion of at least 50%, 60%, 70%, 80%, 90%, 95%, 99% or 99.9% by molar ratio, volume, or weight or mass. The statement that a stream “exclusively” contains one or more components must not, however, exclude the possibility of traces of one or more other components being present. The term “traces” denotes, for example, amounts of less than 1%, 0.1%, 0.01% or 0.001% by molar ratio, volume, or weight or mass. Where there is mention of a “methane stream”, an “ethane stream”, an “ethylene stream” or the like, this refers to a stream which predominantly or exclusively contains the respective component in the sense explained above.

### Advantages of the Invention

The present invention starts from a method for producing hydrocarbons, wherein one or more steam cracking feed streams which predominantly or exclusively contain hydrocarbons with two or more carbon atoms are subjected to one or more steam cracking steps, thereby obtaining one or more steam cracking discharge streams. An apparatus of this kind is shown for example in FIG. 2A described hereinafter. The steam cracking steps carried out in such a process may, as already explained, encompass the use of one or more cracking furnaces operated under identical or different conditions and/or supplied with identical or different feeds. For example, one or more so-called naphtha cracking furnaces (i.e. cracking furnaces for heavier fresh feeds) and one or more so-called gas cracking furnaces (i.e. cracking furnaces for gaseous fresh feeds) may be provided.

Moreover, the method according to the invention comprises subjecting one or more reaction feed streams which predominantly or exclusively contain methane to one or more steps for the oxidative coupling of methane, thereby producing one or more reaction discharge streams which contain ethane. Methods for the oxidative coupling of methane were explained at the beginning and are fundamentally known to the skilled man.

Within the scope of the present invention, a separation discharge stream which predominantly or exclusively contains ethane is formed using fluid from the steam cracking discharge stream or streams. A separation discharge stream of this kind may be produced in known processes or separation sequences, for example using demethanizers and deethanizers and the known C2-splitters, as explained in the above-mentioned article “Ethylene” in Ullmann's Encyclopedia of Industrial Chemistry.

According to the invention, one or more so-called post-bed-cracking steps are provided, i.e. one or more thermal cracking steps, to which fluid from the reaction discharge stream or streams from the oxidative coupling of methane are subjected, and in which the waste heat from the oxidative coupling of methane is used for the cracking. As is fundamentally known to the skilled man, large amounts of waste heat are produced in the oxidative coupling of methane, due to the exothermic nature of the reaction. In the subsequent thermal cracking step or steps, the ethane which is present in the fluid of the reaction discharge stream or streams and which has been formed by the oxidative coupling of methane is reacted at least partially to form ethylene, under the effect of the waste heat from the step or steps for the oxidative coupling of methane. The step or steps for the oxidative coupling of methane and the subsequent thermal cracking step or steps are carried out in a joint reactor. The transfer of heat into the subsequent thermal cracking step or steps takes place by convection, i.e. the heat is transferred with the stream of fluid from a reaction zone for carrying out the step or steps for the oxidative coupling of methane into a reaction zone for carrying out the thermal cracking step or steps.

An essential aspect of the present invention is to supply fluid from the separation discharge stream, which was formed from the fluid of the steam cracking discharge stream, and which predominantly or exclusively contains ethane, to the thermal cracking step or steps, i.e. to the post-bed-cracking step or steps. In this way, a particularly advantageous material integration can be achieved in a corresponding combined process, namely by further reacting an inherently worthless product of a steam cracking process at a particularly favourable location.

Whereas in conventional plants for processing ethane as a recycle stream or fresh feed, separate steam cracking steps have to be implemented by providing gas cracking furnaces, as explained for example with reference to FIG. 2A, these steps are not required in a combined process as proposed by the present invention. The process according to the invention thus makes it possible to achieve an advantageous material utilisation of the methane produced in the steam cracking step or steps without any great additional costs. Since no separate gas cracking furnace has to be provided, as already mentioned, the process conditions can be kept the same for all the steam cracking steps (typically a number of parallel steam cracking steps are provided in such processes). This reduces the control and/or adjustment costs considerably.

It is particularly advantageous if, within the scope of the present invention, using fluid from the steam cracking discharge stream or streams, another separation discharge stream which predominantly or exclusively contains methane or methane and hydrogen is formed. In this case, fluid from the additional separation discharge stream may be used as the or one of the reaction feed streams. The quantity of the fluid used accordingly depends particularly on the methane requirement of the step or steps for the oxidative coupling of methane. By using the methane in corresponding steps, another material integration and a value-adding utilisation of the methane can be achieved. This is generally more advantageous than burning methane for heat. Likewise, any methane occurring within the scope of the present invention may be used for heating.

Another advantageous embodiment of the process according to the invention comprises forming a stream of pyrolysis oil and/or pyrolysis gasoline, using fluid from the steam cracking discharge stream or streams, while fluid from the stream of pyrolysis oil and/or pyrolysis gasoline may also be used to heat the steam cracking step or steps. In this way it is possible to compensate for a reduced supply of methane when this is fed into the step or steps for the oxidative coupling of methane. Corresponding heating may be carried out directly or indirectly. By direct heating is meant that the fluid from the stream of pyrolysis oil and/or pyrolysis gasoline is combusted, i.e. burned, directly in a steam cracking reactor. It may also be envisaged that steam may be generated by combustion of the fluid from the stream of pyrolysis oil and/or pyrolysis gasoline, this steam may be used to generate electrical energy and to heat the steam cracking step or steps electrically, i.e. indirectly.

Although it was emphasised previously that within the scope of the present invention ethane from the separation discharge stream which predominantly or exclusively contains ethane is advantageously used in the thermal cracking step or steps, fluid from this separation discharge stream may obviously also be subjected to the, or one of the, steam cracking steps, particularly when a supply of ethane exceeds a requirement of the thermal cracking step or steps. In this way, an existing ethane or gas cracking furnace intended to be supplied with a recycled ethane stream can be supplied, for example, and/or the fluid may be supplied to another steam cracking feed stream.

In the latter case it has proved particularly advantageous if a quantity of the fluid from the separation discharge stream, which is fed into the thermal cracking step or steps, and/or a quantity of the fluid from the separation discharge stream which is subjected to the or one of the steam cracking steps, is adjusted as a function of an ethane requirement of the thermal cracking step or steps and/or the steam cracking step or steps. For this purpose, suitable control and/or adjustment means may be provided which allow a particularly flexible management of the process, adapted to the particular requirements.

It is particularly advantageous if fluid from the steam cracking discharge stream or streams and fluid from one or more discharge streams of the thermal cracking step or steps, which follow the step or steps for the oxidative coupling of methane, are subjected to at least one joint processing and/or separation step. As basically chemically comparable compounds are present in the steam cracking discharge stream or streams and the discharge stream or streams of the thermal cracking step or steps, joint separation has particular advantages and makes it possible to dispense with redundant separation equipment.

It is particularly advantageous if the waste heat from the step or steps for the oxidative coupling of methane is used in a joint reactor, in which the thermal cracking step or steps is or are also carried out. Corresponding reactors comprise a catalysis zone which is arranged for carrying out the oxidative coupling of methane. Following this catalysis zone, in a joint construction unit, is a zone in which the waste heat from the oxidative coupling of methane can act on the ethane formed. Within the scope of the present invention, the ethane from the separation discharge stream is fed into this subsequent zone.

The present invention also relates to an apparatus for the production of hydrocarbons. An apparatus of this kind comprises means which are designed to subject one or more steam cracking feed streams which predominantly exclusively contain hydrocarbons with two or more carbon atoms to one or more steam cracking steps, thereby obtaining one or more steam cracking discharge streams. Moreover, these means are designed to subject one or more reaction feed streams which predominantly or exclusively contain methane to one or more steps for the oxidative coupling of methane, thus obtaining one or more reaction discharge streams which contain ethane. In addition, means are provided which are designed to form a separation discharge stream which predominantly or exclusively contains ethane, using fluid from the steam cracking discharge stream or streams.

According to the invention, an apparatus of this kind is characterised by means which are designed to subject fluid from the reaction feed stream or streams to one or more thermal cracking steps which follow the step or steps for the oxidative coupling of methane, and to at least partially react the ethane which is present in the fluid of the reaction discharge stream or streams to form ethylene, under the influence of waste heat from the step or steps for the oxidative coupling of methane. Moreover, means are provided which are designed to supply fluid from the separation discharge stream to the thermal cracking step or steps. An apparatus of this kind enjoys the advantages explained hereinbefore, to which reference is therefore expressly made. In particular, an apparatus of this kind comprises one or more joint reactors as previously explained, which are designed to carry out the step or steps for oxidative coupling of methane and the thermal cracking step or steps.

A particularly advantageous apparatus is designed to carry out a process as described in detail hereinbefore.

The invention is hereinafter explained in more detail with reference to the drawings which show a preferred embodiment of the invention.

### BRIEF DESCRIPTION OF THE DRAWINGS

FIG. 1 shows a method for producing hydrocarbons according to one embodiment of the invention, in the form of a schematic flow diagram.

FIGS. 2A and 2B illustrate the conversion of a method according to one embodiment of the invention in the form of a schematic flow diagram.

### DETAILED DESCRIPTION OF THE DRAWINGS

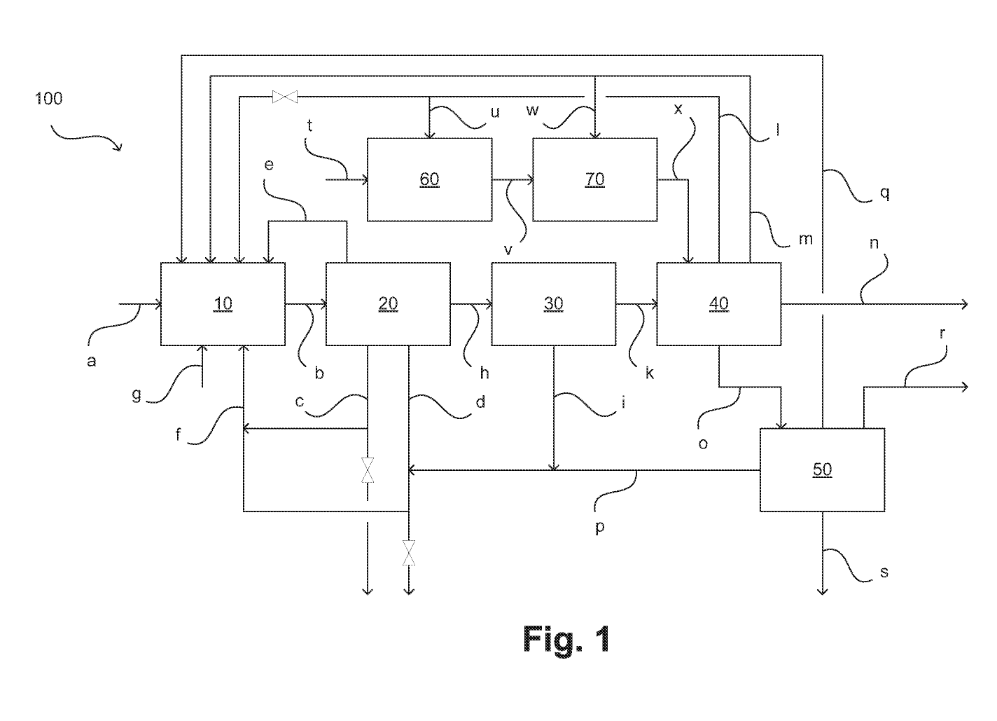

FIG. 1 shows a method for producing hydrocarbons according to one embodiment of the invention in the form of a schematic flow diagram, which is generally designed 100.

One or more steam cracking steps, which have been supplied with one or more steam cracking feed streams a, are designated 10 here. One or more steam cracking discharge streams b formed in the steam cracking step or steps 10 are initially fed into an oil and water wash 20, whereby first of all a (crude) pyrolysis oil stream c and a (crude)pyrolysis gasoline stream d, for example, are separated off. A steam stream e may be fed back into the steam cracking step or steps 10. The pyrolysis oil stream c and the pyrolysis gasoline stream d may be prepared in any desired manner and separated into fractions. It is also possible to obtain a stream f, which is thermally utilised to recover heat energy at least intermittently in the steam cracking step or steps 10. Additionally or exclusively, heat energy may be provided, at least intermittently, through another combustible stream g. Fractions of the streams c and d may be taken out of the process 200 as products.

A stream h remaining after the oil and water wash 20 may be subjected to a compression and drying 30, at which point another pyrolysis gasoline stream i may be obtained. A stream k remains, which can be subjected to a low temperature separation 40. In the low temperature separation 40, a number of separation discharge streams are obtained, such as for example a fuel gas stream 1, an ethane stream m, an ethylene stream n and a stream o of hydrocarbons with three or more carbon atoms. The stream o is subjected to a further separation 50, in which another pyrolysis gasoline stream p, a propane stream q, a propylene stream r and a stream s of hydrocarbons with four carbon atoms may be obtained.

The fuel gas stream 1, the ethane stream m and the propane stream q are conventionally recycled into the steam cracking step or steps 10, while the fuel gas stream 1 is conventionally combusted.

The method 100 shown in FIG. 1 comprises one or more steps 60 for the oxidative coupling of methane into which one or more reaction feed streams t are fed. A stream u formed from fluid from the fuel gas stream m may also be fed, as a reaction feed stream, into the step or steps 60 for the oxidative coupling of methane. The step or steps 60 for the oxidative coupling of methane operate in a manner known per se, so as to form one or more reaction discharge streams v. Additional streams fed into the step or steps 60 for the oxidative coupling of methane, for example one or more oxygen streams, are not shown in the drawings.

The step or steps 60 for the oxidative coupling of methane are directly followed by one or more thermal cracking steps 70 (so-called post-bed cracking). As previously mentioned, a large amount of waste heat is produced during the oxidative coupling of methane, which can advantageously be used, for example, for cracking the ethane present during the oxidative coupling of methane. The amount of waste heat is so great that a larger amount of ethane can be cracked than is formed during the oxidative coupling of methane. Although the thermal cracking step or steps 70 in FIG. 1 are shown separately from the step or steps 60 for the oxidative coupling of methane, it should be remembered that these are typically implemented in a single construction unit.

Within the scope of the embodiment of the invention shown in FIG. 1, it is envisaged that fluid from the ethane stream m, i.e. a corresponding separation discharge stream from the low temperature separation 40, may be fed into the thermal cracking step or steps 70. Corresponding fluid is shown as the stream w. In this way, the ethane can be profitably used and there is no need for externally supplied ethane. Likewise, if necessary, some of the stream m may also be recycled into the steam cracking step or steps 10 or external ethane may be fed in.

A discharge stream x from the thermal cracking step or steps 70 may for example be guided into the low temperature separation 40. It is also possible to feed it into other steps, for example into the compression and drying 30.

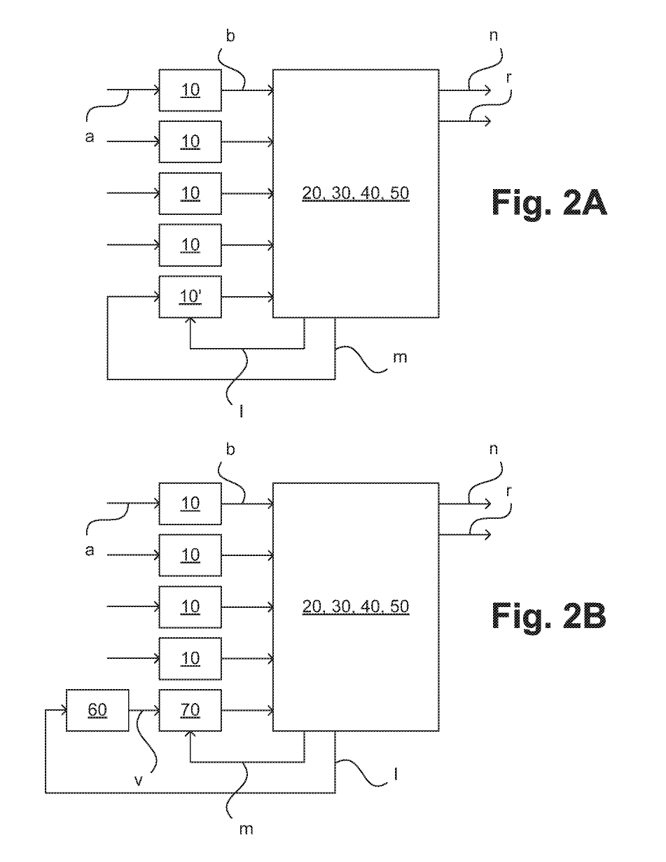

FIGS. 2A and 2B illustrate the conversion of a method according to one embodiment of the invention in a pure steam cracking process in the form of a schematic flow diagram. The streams shown are given identical reference numerals to the streams in FIG. 1. FIGS. 2A and 2B are also highly simplified.

FIG. 2A shows, for example, an existing apparatus for the production of hydrocarbons, in which five parallel steam cracking steps are implemented. The steam cracking steps designated 10 are provided, for example, for reacting steam cracking feeds a (identified only at one point) which contain a large proportion of naphtha. In each case, steam cracking discharge streams b are obtained (identified only at one point). A steam cracking step designated 10′, on the other hand, is provided for reacting steam cracking feeds which predominantly or exclusively contain ethane (so-called gas cracking furnace).

The steam cracking discharge streams b are fed into a processing and separation step, shown highly schematically here, which comprises steps 10 to 50 shown in FIG. 1, for example. In addition to other separation discharge streams, an ethylene stream n and a propylene stream r are obtained, for example, and removed as products. An ethane stream m which is also obtained as a separation discharge stream is introduced into the steam cracking step designated 10′, where it is reacted. A methane stream 1 which has also been obtained as a separation discharge stream is used to heat the steam cracking step designated 10′.

According to FIG. 2B, the steam cracking step designated 10′ is replaced by one or more steps 60 for the oxidative coupling of methane, into which the methane stream 1 is wholly or partially fed, among other things. The ethane stream is wholly or partially fed into one or more subsequent thermal cracking steps 70, as explained hereinbefore.

1. Method (100) for producing hydrocarbons, wherein one or more steam cracking feed streams (a) which predominantly or exclusively contain hydrocarbons with two or more carbon atoms are subjected to one or more steam cracking steps (10), thus obtaining one or more steam cracking discharge streams (b), and wherein one or more reaction feed streams (t, u) which predominantly or exclusively contain methane are subjected to one or more steps (60) for the oxidative coupling of methane, thus obtaining one or more reaction discharge streams (v) which contain ethane, while a separation discharge stream (m) which predominantly or exclusively contains ethane is formed using fluid from the steam cracking discharge stream or streams (b), characterised in that fluid from the reaction discharge stream or streams (v) is subjected to one or more thermal cracking steps (70) which are subsequent to the step or steps (60) for the oxidative coupling of methane, and in which the ethane which is present in the fluid from the reaction discharge stream or streams (v) is at least partially reacted to form ethylene, under the influence of waste heat from the step or steps (60) for the oxidative coupling of methane, and in that fluid (w) from the separation discharge stream (m) is fed into the thermal cracking step or steps (70), wherein the step or steps (60) for the oxidative coupling of methane and the subsequent thermal cracking step or steps (70) are carried out in a joint reactor and wherein the transfer of heat into the thermal cracking step or steps (70) that follow takes place by convection.

2. Method (100) according to claim 1, wherein an additional separation discharge stream (l) which predominantly or exclusively contains methane or methane and hydrogen is formed using fluid from the steam cracking discharge stream or streams (b), wherein fluid (u) from the additional separation discharge stream (l) is used as the or one of the reaction feed streams.

3. Method (100) according to one of the preceding claims, wherein a pyrolysis oil (c) and/or a pyrolysis gasoline stream (d) is formed using fluid from the steam cracking discharge stream or streams (b), while fluid from the pyrolysis oil (c) and/or the pyrolysis gasoline stream (d) is used to heat the steam cracking step or steps (10).

4. Method (100) according to one of the preceding claims, wherein fluid from the separation discharge stream (m) is subjected to the or one of the steam cracking steps (10′).

5. Method (100) according to claim 4, wherein a quantity of the fluid (w) from the separation discharge stream (m) which is fed into the thermal cracking step or steps (70), and/or a quantity of the fluid from the separation discharge stream (m) which is subjected to the or one of the steam cracking steps (10′), is adjusted as a function of an ethane requirement of the thermal cracking step or steps (70) and/or of the steam cracking step or steps (10′).

6. Method (100) according to one of the preceding claims, wherein fluid from the steam cracking discharge stream or streams (b) and fluid from one or more discharge streams (x) of the thermal cracking step or steps (70) which follow the step or steps (60) for the oxidative coupling of methane, is subjected to at least one joint processing and/or separation step (20-50).

7. Method (100) according to one of the preceding claims, wherein all the fluid from the reaction discharge stream or streams (v) is subjected, without separation, to the one or more thermal cracking steps (70) which follow the step or steps (60) for the oxidative coupling of methane.

8. Method (100) according to one of the preceding claims, wherein the waste heat from the step or steps (60) for the oxidative coupling of methane is used in a joint reactor in which the thermal cracking step or steps (70) are carried out.
